(19) United States
(12) Patent Application Publication
(10) Pub. No.: US 2025/0261296 A1
(43) Pub. Date: Aug. 14, 2025

(54) X-RAY DEVICE AND IMAGING DEVICE INCLUDING X-RAY DEVICE

(71) Applicant: WUHAN UNITED IMAGING HEALTHCARE CO., LTD., Wuhan, Hubei (CN)

(72) Inventors: Yaoqiang DUAN, Wuhan (CN); Zhi CHEN, Wuhan (CN); Tieshan ZHANG, Shanghai (CN); Weihua LI, Wuhan (CN); Xu CHU, Shanghai (CN)

(73) Assignee: WUHAN UNITED IMAGING HEALTHCARE CO., LTD., Wuhan, Hubei (CN)

(21) Appl. No.: 19/192,307

(22) Filed: Apr. 28, 2025

Related U.S. Application Data
(63) Continuation of application No. PCT/CN2023/127887, filed on Oct. 30, 2023.

(30) Foreign Application Priority Data
Oct. 28, 2022 (CN) 202211338073.2

(51) Int. Cl. H05G 1/12; G01N 23/046; H05G 1/06; H05G 1/20

(52) U.S. Cl. CPC H05G 1/12; G01N 23/046; H05G 1/06; H05G 1/20

(57) ABSTRACT

Embodiments of the present disclosure provide an X-ray device and an imaging device including the X-ray device. The X-ray device may include a high voltage generator and an X-ray tube. The high voltage generator and the X-ray tube may be disposed in a same housing. The housing may be filled with an insulating material.

### CROSS-REFERENCE TO RELATED APPLICATIONS

This application is a continuation of International Patent Application No. PCT/CN2023/127887, filed on Oct. 30, 2023, which claims priority of Chinese Patent Application No. 202211338073.2, filed on Oct. 28, 2022, the entire contents of which are incorporated herein by reference.

### TECHNICAL FIELD

The present disclosure relates to the technical field of medical devices, and in particular, to an X-ray device and an imaging device including the X-ray device.

### BACKGROUND

An X-ray imaging device (e.g., a Digital Radiography (DR) device or a Computed Tomography (CT) device) includes a high voltage generator, an X-ray tube, and a detector. The high voltage generator is used to convert a low-voltage into a direct current (DC) high-voltage and applies the DC high-voltage between an anode and a cathode of the X-ray tube to generate a high-voltage electric field between the anode and the cathode of the X-ray tube. At the same time, the high voltage generator provides a power supply for a filament of a cathode of the X-ray tube, so that the filament heats up to generate free electrons at the cathode, and the free electrons impact an anode target disk at a high speed under the control of the high-voltage electric field to generate X-rays. A connection structure (e.g., a high-voltage connection device or a length of a high-voltage cable) between the high voltage generator and the tube not only affects ease of operation of the imaging device, but also affects work efficiency of the imaging device. Additionally, the high voltage connection device has a problem such as high costs and unreliable connection.

Therefore, it is desirable to provide an X-ray device that reduces the complexity of a connection between the high voltage generator and the X-ray tube, thereby saving the cost.

### SUMMARY

One or more embodiments of the present disclosure provide an X-ray device. The X-ray device may include a high voltage generator and an X-ray tube. The high voltage generator and the X-ray tube may be disposed in a same housing. The housing may be filled with an insulating material.

One or more embodiments of the present disclosure provide an imaging device. The imaging device may include an X-ray device. The X-ray device may include a high voltage generator and an X-ray tube. The high voltage generator and the X-ray tube may be disposed in a same housing. The housing may be filled with an insulating material.

### BRIEF DESCRIPTION OF THE DRAWINGS

The present disclosure is further illustrated in terms of exemplary embodiments. These exemplary embodiments are described in detail with reference to the drawings. These embodiments are non-limiting exemplary embodiments, in which the same reference numbers represent the same structures, wherein:

FIG. 1 is a schematic diagram illustrating a power transfer chain of a Computed Tomography (CT) device in the prior art;

FIG. 2 is a schematic diagram illustrating a connection between a high voltage generator and a tube in FIG. 1;

FIG. 3 is a block diagram illustrating an exemplary structure of an X-ray device according to some embodiments of the present disclosure;

FIG. 4 is a schematic diagram illustrating an exemplary circuit connection between a filament control unit and a filament transformer according to some embodiments of the present disclosure;

FIG. 5 is a schematic diagram illustrating an exemplary circuit structure of an X-ray device corresponding to a bipolar tube according to some embodiments of the present disclosure;

FIG. 6 is a schematic diagram illustrating an exemplary circuit structure of an X-ray device corresponding to a unipolar tube according to some embodiments of the present disclosure;

FIG. 7 is a schematic diagram illustrating an exemplary three-dimensional structure of a planar transformer according to some embodiments of the present disclosure;

FIG. 8 is a side view of a planar transformer according to some embodiments of the present disclosure;

FIG. 9(a) is a schematic diagram illustrating an exemplary circuit structure of an X-ray device according to some embodiments of the present disclosure;

FIG. 9(b) is a schematic diagram illustrating an exemplary circuit structure of an X-ray device according to some embodiments of the present disclosure;

FIG. 10 is a schematic diagram illustrating an exemplary structure of a rectifier board according to some embodiments of the present disclosure;

FIG. 11 is a schematic diagram illustrating an exemplary connection structure between a rectifier board and a boost transformer according to some embodiments of the present disclosure;

FIG. 12 is a three-dimensional sectional schematic diagram illustrating an exemplary internal structure of an X-ray device according to some embodiments of the present disclosure;

FIG. 13 is a three-dimensional sectional schematic diagram illustrating an exemplary internal structure of an X-ray device according other embodiments of the present disclosure;

FIG. 14 is a block diagram illustrating an exemplary structure of an imaging device according to some embodiments of the present disclosure;

FIG. 15 is a schematic diagram illustrating an exemplary connection of various components of an imaging device according to some embodiments of the present disclosure;

FIG. 16 is a schematic diagram illustrating an exemplary connection between an anode driver and a motor according to some embodiments of the present disclosure;

FIG. 17 is a schematic diagram illustrating an exemplary circuit structure of an imaging device according to some embodiments of the present disclosure; and

FIG. 18 is a schematic diagram illustrating an exemplary circuit structure of an imaging device according to other embodiments of the present disclosure.

### DETAILED DESCRIPTION

In order to more clearly illustrate the technical solutions related to the embodiments of the present disclosure, a brief introduction of the drawings referred to the description of the embodiments is provided below. Obviously, the drawings described below are only some examples or embodiments of the present disclosure. Those having ordinary skills in the art, without further creative efforts, may apply the present disclosure to other similar scenarios according to these drawings. Unless obviously obtained from the context or the context illustrates otherwise, the same numeral in the drawings refers to the same structure or operation.

It should be understood that the “system,” “device,” “unit,” and/or “module” used herein are one method to distinguish different components, elements, parts, sections, or assemblies of different levels. However, if other words can achieve the same purpose, the words can be replaced by other expressions.

As used in the disclosure and the appended claims, the singular forms “a,” “an,” and “the” include plural referents unless the content clearly dictates otherwise; the plural forms may be intended to include singular forms as well. In general, the terms “comprise,” “comprises,” and/or “comprising,” “include,” “includes,” and/or “including,” merely prompt to include steps and elements that have been clearly identified, and these steps and elements do not constitute an exclusive listing. The methods or devices may also include other steps or elements.

The flowcharts used in the present disclosure illustrate operations that the system implements according to the embodiment of the present disclosure. It should be understood that the foregoing or following operations may not necessarily be performed exactly in order. Instead, the operations may be processed in reverse order or simultaneously. Besides, one or more other operations may be added to these processes, or one or more operations may be removed from these processes.

An X-ray imaging device (e.g., a Digital Radiography (DR) device or a Computed Tomography (CT) device) may collect scanning data related to a target object by emitting X-rays to the target object and collecting X-rays that pass through the target object using a detector, thereby achieving scanning of the target object. The target object may be biological or non-biological. For example, the target object may include a patient, a man-made object (e.g., a man-made mold), etc. As another example, the target object may include a specific part, an organ, and/or tissue (e.g., a head, ears, a nose, a mouth, a neck, a chest, an abdomen, a liver, a gallbladder, a pancreas, a spleen, a kidney, a spine, a heart, or tumor tissue) of the patient.

X-rays, also known as roentgen rays, have an ability to penetrate a substance, but the X-rays have different penetrating abilities for different substances. Human tissue has a difference in density and thickness. Due to the difference, when X-rays pass through a plurality of different tissue structures of a human body, X-rays are absorbed to different extents, so amounts of X-rays that reach a screen or film are different. In this way, the X-rays may form black and white images with different contrasts on the screen or film. Therefore, the X-ray imaging equipment has a great use value in basic technology and in new clinical applications.

A main component of an image chain of the X-ray imaging device may include a high voltage generator (HVG), an X-ray tube (referred to as a tube), and a detector. The tube may be used to emit X-rays to the target object. The detector may be used to receive X-rays passing through the target object. The HVG may be used to convert a power frequency alternating current (AC) voltage into a direct current (DC) high-voltage (up to 140 kV˜150 kV) and apply the DC high-voltage between an anode and a cathode of the tube, thereby generating a high-voltage electric field between the anode and the cathode of the tube. At the same time, the HVG may provide a control power for a filament of the cathode of the tube, so that the filament may heat up to generate free electrons at the cathode, and the free electrons may impact an anode target disk of the tube at a high speed under the control of the high-voltage electric field to generate X-rays.

However, in the imaging chain of a conventional X-ray imaging device, the high voltage generator and the tube are two structurally independent power components. A high-voltage connection device (e.g., a high-voltage cable or a high-voltage socket) between the high voltage generator and the tube may be not only expensive, but also have a risk of unreliable high-voltage connection, which may not only affect ease of operation of the imaging device, but also affect work efficiency and service life of the imaging device. For example, in the DR device, the high-voltage cable connecting the high voltage generator to the tube may be tens of meters long, which affects simplicity of appearance of the entire DR device and the ease of motion operation. As another example, in a rotating gantry of the CT device, the high voltage generator may usually include an inverter and a high voltage oil tank, and the high voltage generator not only occupies a large portion of space of the rotating gantry but also weighs a lot, which exacerbates wear and tear of a bearing of the rotating gantry of the CT device and may be not conducive to increasing a speed of the rotating gantry.

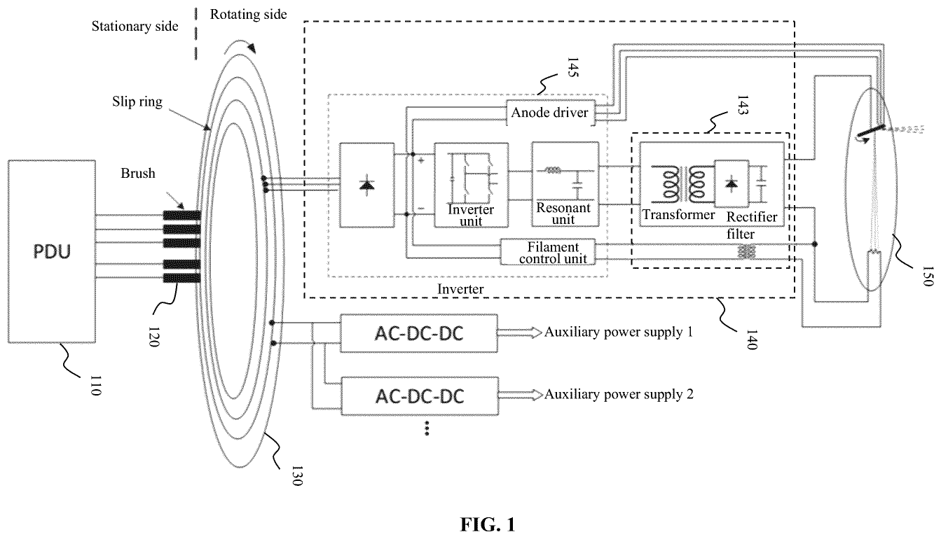

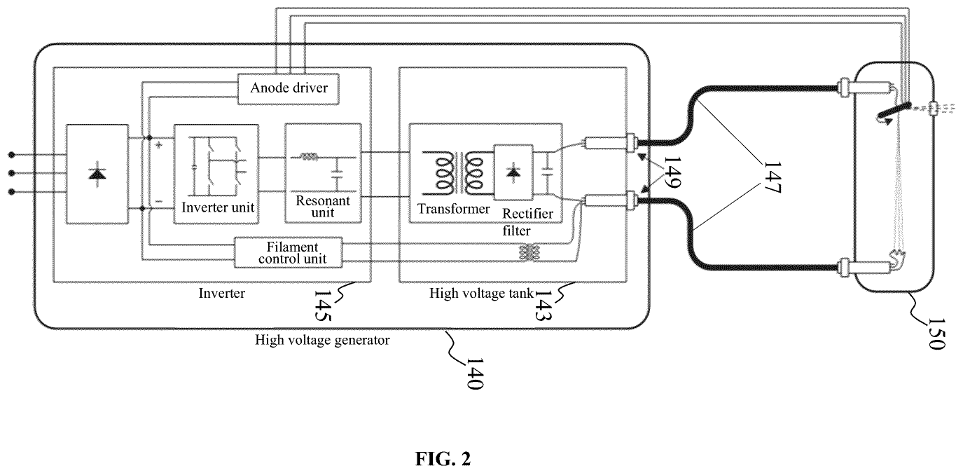

As described in connection with FIG. 1 and FIG. 2, taking a CT device as an example, an imaging chain of an X-ray imaging device is illustrated as follows.

FIG. 1 is a schematic diagram illustrating a power transfer chain of a CT device in the prior art. As illustrated in FIG. 1, the power transfer chain of the CT device may include a Power Distribution Unit (PDU) 110, a brush 120, a slip ring 130, a high voltage generator (HVG) 140, an X-ray tube 150, and other auxiliary control system units (e.g., auxiliary power supply 1 or auxiliary power supply 2). In some embodiments, the power transfer chain of the CT device may also include a detector array (not shown). In some embodiments, the HVG 140 may also include an inverter unit and a resonant unit.

The PDU 110 and the brush 120 may be located on a stationary side of the CT device. An interior of the PDU 110 may mainly include a power frequency isolation transformer and a protection control unit. The slip ring 130, the high voltage generator 140, the X-ray tube 150, and the detector array may be fixed to a rotating gantry of the CT device and rotate together with the gantry. Through contact friction between the brush 120 mounted on the stationary side and mounted on the rotating gantry, power source output by the PDU 110 may be transferred to the gantry to power the component such as the HVG 140 or the detector array.

During operation, the HVG 140 may convert an AC low-voltage into a DC high-voltage and apply the DC high-voltage between an anode and a cathode of the X-ray tube 150, thereby generating a high-voltage electric field between the anode and the cathode of the X-ray tube 150. At the same time, the HVG 140 may provide a power supply for a filament of the cathode of the X-ray tube 150, so that the filament may heat up to generate free electrons at the cathode, and the free electrons may impact an anode target disk of the X-ray tube 150 at a high speed under the control of the high-voltage electric field to generate X-rays. As shown in FIG. 1, the HVG 140 may also power an anode motor of the X-ray tube 150 via an anode driver to make the anode target disk rotate at a high speed.

FIG. 2 is a schematic diagram illustrating a connection between the high voltage generator 140 and the X-ray tube 150 in FIG. 1. As illustrated in FIG. 2, in the CT device in the prior art, the high voltage generator 140 and the X-ray tube 150 may be required to be connected to each other through a high-voltage connection device such as a high-voltage cable 147 and a high-voltage socket 149. In addition, in a rotating gantry of the CT device, the high voltage generator 140 may include an inverter 145 and a high voltage oil tank (also known as a high voltage tank) 143, which not only makes the HVG heavy, but also occupies a large portion of space of the rotating gantry. Additionally, as power of the CT device increases, a diameter and a volume of the rotating gantry may increase, and in order to maintain a dynamic balance of the rotating gantry, it may be necessary to increase a corresponding counterweight on the rotating gantry, so that not only a load-bearing of the rotating gantry is significantly increased, but also a maximum speed of the rotating gantry may be limited due to the relatively large diameter and the load-bearing of the gantry.

Embodiments of the present disclosure provide an X-ray device and an imaging device including the X-ray device. The X-ray device may include a high voltage generator and an X-ray tube disposed in a same housing, and the housing is filled with an insulating material, so that the high voltage generator and the X-ray tube may be in a same insulating environment. As a result, there may be no need to use the high-voltage connection device such as a high-voltage plug, the high-voltage socket, or the high-voltage cable between the high voltage generator including a component such as a boost transformer module or a rectifier filter module and the X-ray tube, which can reduce the complexity of the high-voltage connection and save the cost of the high-voltage connection.

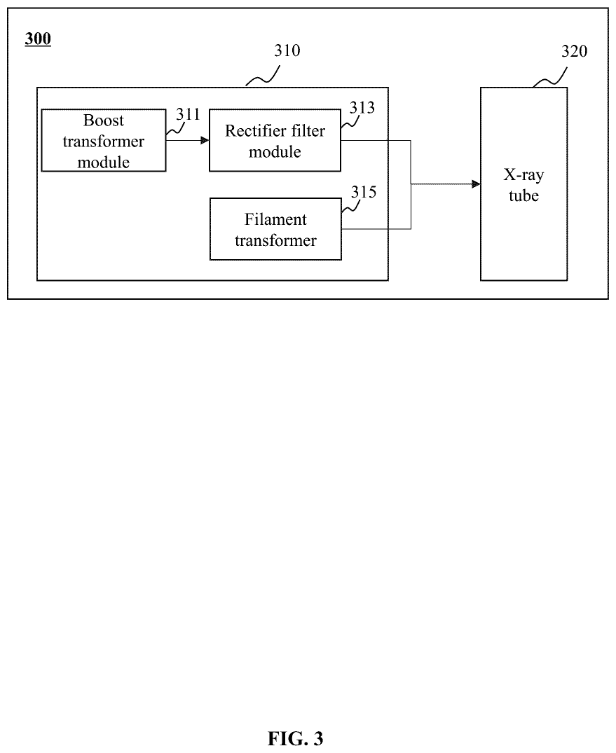

FIG. 3 is a block diagram illustrating an exemplary structure of an X-ray device according some embodiments of the present disclosure. In some embodiments, as shown in FIG. 3, the X-ray device 300 may include a high voltage generator 310 and an X-ray tube 320. The high voltage generator 310 and the X-ray tube 320 may be disposed in a same housing and the housing may be filled with an insulating material.

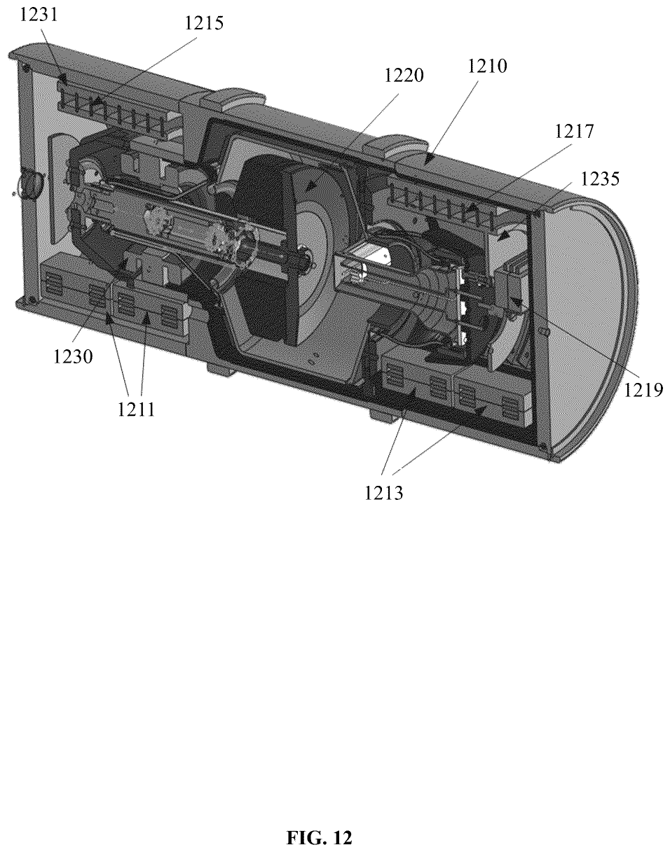

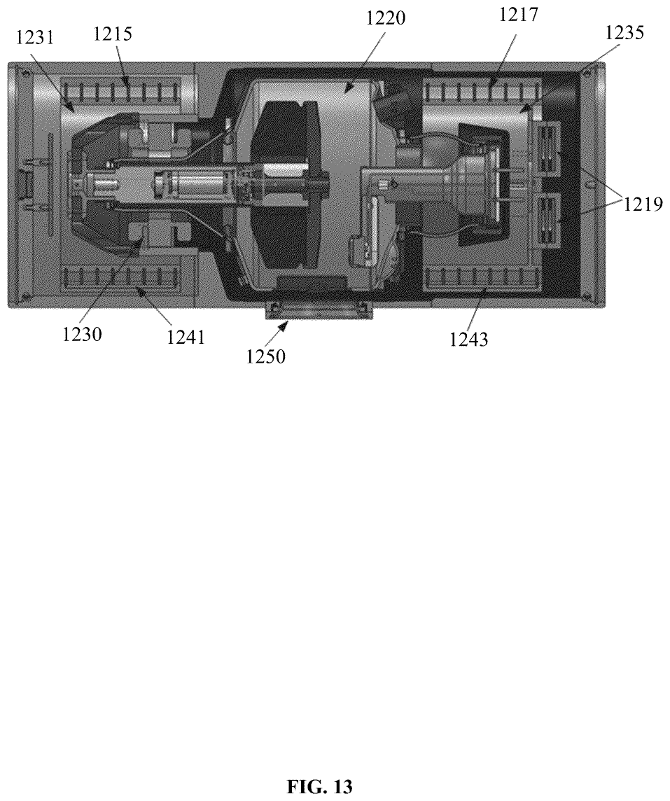

The housing refers to a housing of the X-ray device. In some embodiments, the housing may also be referred to as a cavity. In some embodiments, the insulating material filled in the housing may include an insulating oil (e.g., a natural mineral oil, a natural vegetable oil, or a synthetic oil), an insulating varnish, an insulating adhesive, an insulating gas (e.g., sulfur hexafluoride), etc. In some embodiments, the insulating material may be filled in an inner wall and/or an outer wall of the housing. In some embodiments, the housing may also include a solid insulating material for insulation and structural support. For example, a solid insulating member may be disposed between the X-ray tube and the rectifier filter module, which may be used to support a rectifier filter structure and insulate the X-ray tube and the rectifier filter module. More descriptions regarding the insulating member may be found in the related descriptions of FIG. 12 or FIG. 13.

In some embodiments, the high voltage generator 310 may include at least one of a boost transformer module, a rectifier filter module, and a filament transformer. For example, when the X-ray tube 320 is unipolar, and a cathode of the X-ray tube 320 is grounded (i.e., the X-ray tube 320 outputs an anode high voltage), the high voltage generator 310 may include a boost transformer module 311 and a rectifier filter module 313. The boost transformer module 311, the rectifier filter module 313, and the X-ray tube 320 may be located in the same housing filled with the insulating material. As another example, when the X-ray tube 320 is bipolar, or the X-ray tube 320 is unipolar but an anode of the X-ray tube 320 is grounded (i.e., the X-ray tube 320 outputs a cathode high voltage), the high voltage generator 310 may include the boost transformer module 311, the rectifier filter module 313, and a filament transformer 315. The boost transformer module 311, the rectifier filter module 313, the filament transformer 315, and the X-ray tube 320 may be located in the same housing filled with the insulating material.

The boost transformer module may be configured to receive an AC voltage and increase the AC voltage. The rectifier filter module may be configured to output a DC voltage by rectifying and filtering the increased AC voltage and apply the DC voltage to the X-ray tube. For example, as shown in FIG. 3, the boost transformer module 311, the rectifier filter module 313, and the X-ray tube 320 may be electrically connected in turn. The boost transformer module 311 may increase the received AC voltage and transmit the increased AC voltage to the rectifier filter module 313. The rectifier filter module 313 may output the DC voltage by rectifying and filtering the increased AC voltage and apply the DC voltage to the X-ray tube 320.

In some embodiments, the boost transformer module may include at least one boost transformer, for example, the boost transformer module may include 1 boost transformer, 2 boost transformers, 3 boost transformers, 4 boost transformers, 5 boost transformers, or 6 boost transformers.

In some embodiments, an anode and a cathode of the X-ray tube may be connected to a same count of boost transformers, respectively. For example, the anode and the cathode of the X-ray tube may be connected to 1 boost transformer, 2 boost transformers, or 3 boost transformers, respectively. In some embodiments, the anode and the cathode of the X-ray tube may be connected to different counts of boost transformers, respectively. For example, 1 boost transformer may be connected to a line where the anode of the X-ray tube is located and 2 boost transformers may be connected to a line where the cathode of the X-ray tube is located. As another example, 0 boost transformer may be connected to the line where the anode of the X-ray tube is located, and 1 boost transformer, 2 boost transformers, or 4 boost transformers may be connected to the line where the cathode of the X-ray tube is located.

In some embodiments, when the X-ray tube is bipolar, the boost transformer module may include an even count of boost transformers, such as 2 boost transformers, 4 boost transformers, or 6 boost transformers. Preferably, the lines where the anode and the cathode of the X-ray tube are located may be connected to a same count of boost transformers, respectively. In some embodiments, when the X-ray tube is unipolar, the boost transformer module may include any integer count of boost transformers, such as 1 boost transformer, 2 boost transformers, or 3 boost transformers. In this case, the boost transformer(s) may be connected only to the line where the cathode of the X-ray tube is located, or the boost transformers may be connected to the lines where the cathode and the anode of the X-ray tube are located.

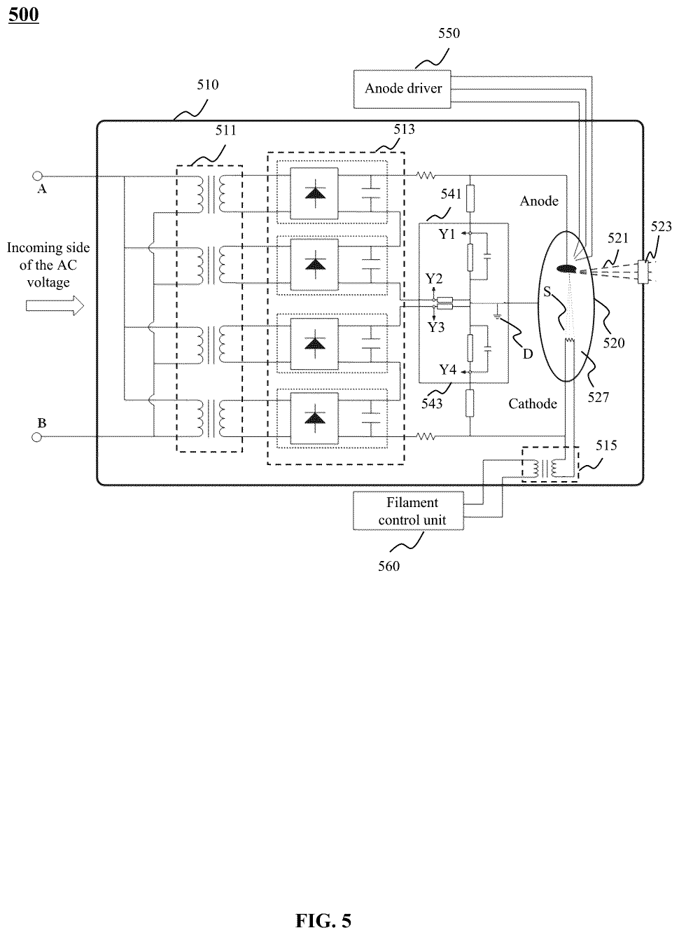

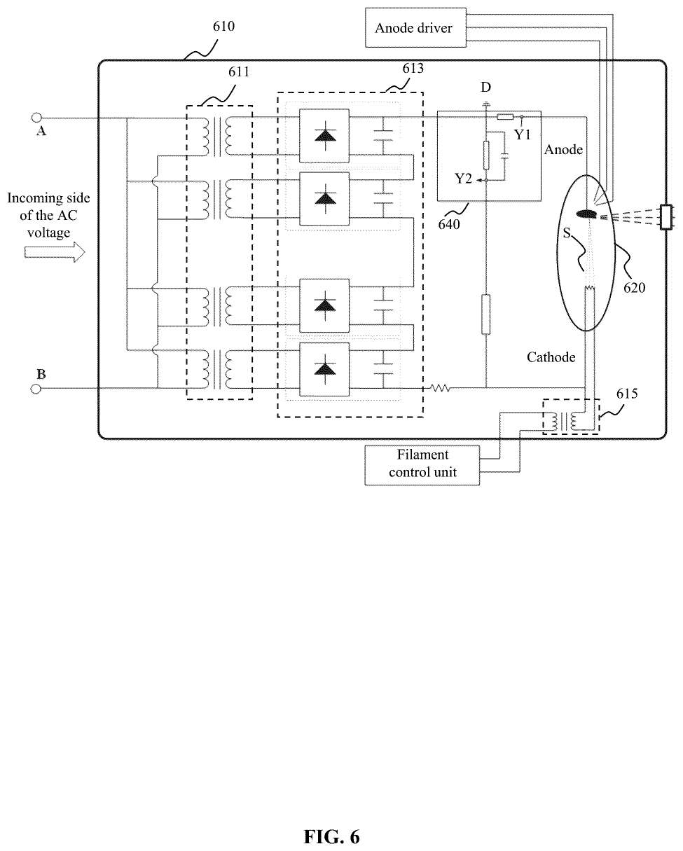

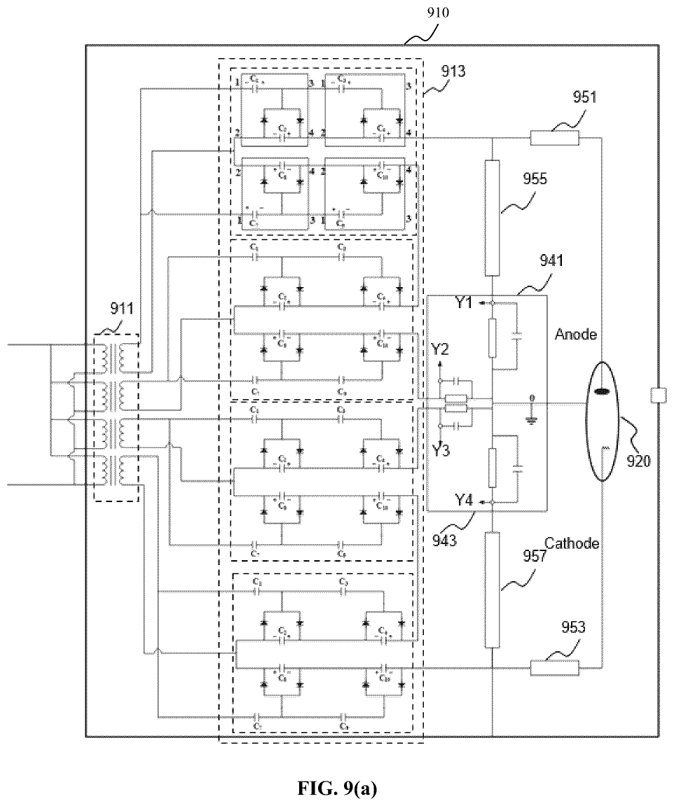

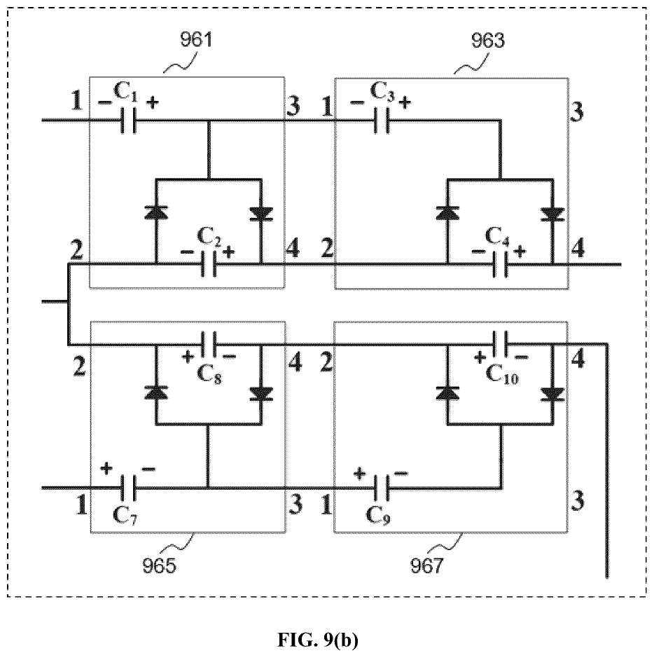

In some embodiments, when the boost transformer module includes two or more boost transformers, a primary side of each of the two or more boost transformers may be connected in parallel, for example, as illustrated in FIG. 5, FIG. 6, FIG. (a), or FIG. 9(b). The primary side refers to a side where an inlet direction of a transmission signal (e.g., current or voltage) is located determined according to a circuit transmission direction (e.g., a flow direction of current and voltage) of the transformer, for example an incoming side of the AC voltage in FIG. 5 or FIG. 6.

In some embodiments, when the boost transformer module includes the two or more boost transformers, the secondary side of each of the two or more boost transformers may be connected in series. The secondary side of each of the two or more boost transformers being connected in series may include that an output end of the secondary side of each boost transformer is connected in series, or an output end of the rectifier filter circuit connected to the secondary side of each boost transformer is connected in series.

In some embodiments, the rectifier filter module may include at least one rectifier filter circuit. For example, the rectifier filter module may include 1 rectifier filter circuit, 2 rectifier filter circuits, 3 rectifier filter circuits, 4 rectifier filter circuits, 5 rectifier filter circuits, or 6 rectifier filter circuits. In some embodiments, when the rectifier filter module includes two or more rectifier filter circuits, output ends of all the rectifier filter circuits may be connected in series, for example, as illustrated in FIG. 5, FIG. 6, FIG. 9(a), or FIG. 9(b). In some embodiments, a count of rectifier filter circuits may be greater than or equal to a count of boost transformers.

In some embodiments, a secondary side of the boost transformer may be connected to an input end of the rectifier filter circuit, for example, as shown in FIG. 5, FIG. 6, FIG. 9(a), or FIG. 9(b). The secondary side refers to a side where an outlet direction of the transmission signal is located determined according to the circuit transmission direction (e.g., the flow direction of current and voltage) of the transformer.

In some embodiments, when the boost transformer module includes the two or more boost transformers, the secondary side of each of the at least two boost transformers may be connected to the input end of one or more of the at least one rectifier filter circuit. For example, the secondary side of each of the boost transformers may be connected to the input end of the rectifier filter circuit corresponding to the boost transformer. As another example, the secondary side of each of the boost transformers may be connected to the input ends of two or more rectifier filter circuits. As yet another example, one of the two boost transformers may be connected to the input ends of two or more rectifier filter circuits, and another of the two boost transformers may be connected to the input end of one rectifier filter circuit.

In some embodiments, an internal circuit connection structure of the rectifier filter module corresponding to the unipolar X-ray tube may be different from an internal circuit connection structure of the rectifier filter module corresponding to the bipolar X-ray tube.

In some embodiments, when the X-ray tube is unipolar, one of the output ends of the at least one rectifier filter circuit may be connected to the anode of the X-ray tube, and another of the output ends of the at least one rectifier filter circuit may be connected to the cathode of the X-ray tube. For example, as shown in FIG. 6, when the X-ray tube is unipolar, one (e.g., a first output end) of the series-connected output ends of the at least one rectifier filter circuit may be connected to an anode of an X-ray tube 620, and another (e.g., a second output end) of the series-connected output ends of the at least one rectifier filter circuit may be connected to an cathode of the X-ray tube 620.

In some embodiments, when the rectifier filter module includes the two or more rectifier filter circuits, an intermediate contact of the two or more rectifier filter circuits may be connected to a ground terminal. Meanwhile, one of the series-connected output ends of all the rectifier filter circuits may be connected to the anode of the X-ray tube and another of the series-connected output ends of all the rectifier filter circuits may be connected to the cathode of the X-ray tube. In some embodiments, when the X-ray tube is bipolar, the intermediate contact of the two or more the rectifier filter circuits may be connected to the ground terminal. One of the series-connected output ends of all of the rectifier filter circuits may be connected to the anode of the X-ray tube and another of the series-connected output ends of all of the rectifier filter circuits may be connected to the cathode of the X-ray tube, for example, as shown in FIG. 5, FIG. 9(a), or FIG. 9(b). It is worth noting that the intermediate contact herein refers to a physically intermediate point of the circuit, e.g., a voltage or current midpoint in the circuit.

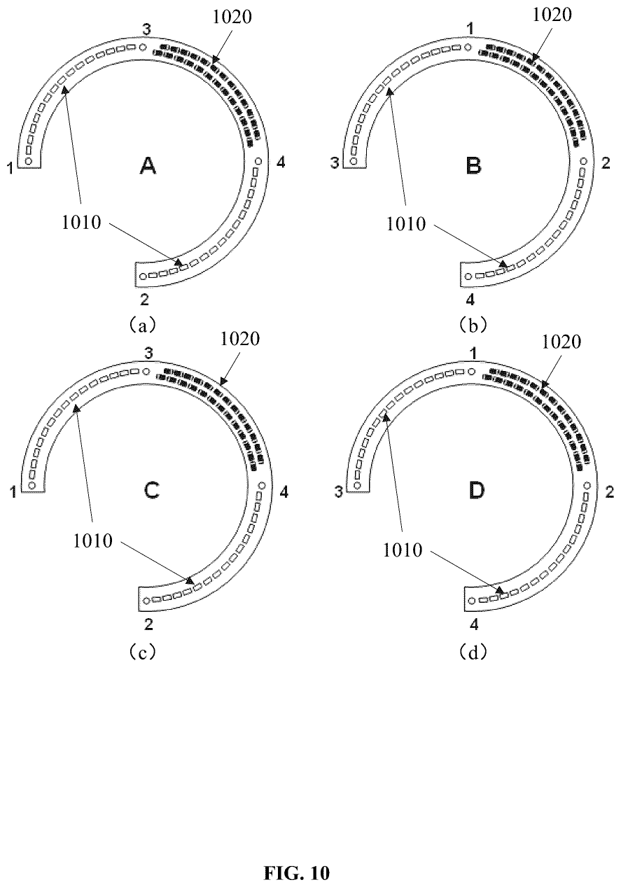

In some embodiments, part or all of each of the rectifier filter circuits may be located on a printed circuit board (PCB). The PCB provided with the rectifier filter circuit may also be referred to as a rectifier board. For example, the rectifier board may include a plurality of diodes and/or capacitors. In some embodiments, the plurality of diodes of the rectifier board may be connected in series using a plurality of surface mounted diodes or In-line diodes. In some embodiments, the plurality of capacitors of the rectifier board may be connected in series using a plurality of surface-mounted multilayer ceramic capacitors or In-line capacitors. In some embodiments, the diodes and capacitors of the rectifier board may be distributed between a plurality of ports, as shown in FIGS. 10(a)˜(d).

In some embodiments, part or all of each of the rectifier filter circuits may be located on a same PCB or different PCBs. For example, two or more rectifier filter circuits included in the rectifier filter module may be located on the same PCB. As another example, the two or more rectifier filter circuits included in the rectifier filter module may be located on the different PCBs, respectively. As yet another example, at least two of the two or more rectifier filter circuits included in the rectifier filter module may be located on the same PCB. Preferably, different rectifier filter circuits may be located on different PCBs.

In some embodiments, different units of one rectifier filter circuit may be located on the same PCB or different PCBs. For example, a plurality of basic units (e.g., four basic units of a bipolar continuous wave (CW) circuit) of the rectifier filter circuit may be located on the same PCB, the plurality of basic units may be located on a plurality of different PCBs, or two or more of the plurality of basic units may be located on the same PCB board. For example, basic unit A of the rectifier filter circuit may be located on PCB 1, basic unit B may be located on PCB 2, basic unit C may be located on PCB 3, and basic unit D may be located on PCB 4. As another example, basic units A and B of the rectifier filter circuit may be located on PCB 1, and basic units C and D may be located on another PCB 2.

In some embodiments, the X-ray device may include at least two PCBs, and the at least two PCBs may be stacked. For example, the different rectifier filter circuits may be located on different PCBs, and all PCBs may be stacked. As another example, the plurality of basic units of the rectifier filter circuit may be located on the different PCBs, and all PCBs may be stacked.

In some embodiments, when the X-ray device includes the at least two PCBs, and the at least two PCBs are stacked, a first PCB may surround the anode of the X-ray tube, and a second PCB may surround the cathode of the X-ray tube, as shown in FIG. 12 or FIG. 13. The first PCB may include a PCB where a first rectifier filter circuit of the at least two rectifier filter circuits is located, and the first rectifier filter circuit may be connected in series between the anode and the ground terminal of the X-ray tube. The second PCB may include a PCB where a second rectifier filter circuit of the at least two rectifier filter circuits is located, and the second rectifier filter circuit may be connected in series between the cathode and the ground terminal of the X-ray tube.

In some embodiments, a second insulating structure may be disposed between the at least two stacked PCBs. For example, the second insulating structure may include polycarbonate (PC) polypropylene, an insulating adhesive, an insulating varnish, etc. In some embodiments, a solid insulating structure may be disposed between the at least two stacked PCBs. In some embodiments, the PCBs may surround the anode or the cathode of the X-ray tube together with an insulating structure corresponding to the PCBs. For example, taking the bipolar X-ray tube as an example, the second insulating structure may include a first insulating member, a second insulating member, a third insulating member, and a fourth insulating member. The first PCB may be disposed between the first insulating member and the second insulating member. The first PCB, the first insulating member, and the second insulating member may all surround the anode of the X-ray tube. The second PCB may be disposed between the third insulating member and the fourth insulating member. The second PCB, the third insulating member, and the fourth insulating member may all surround the cathode of the X-ray tube.

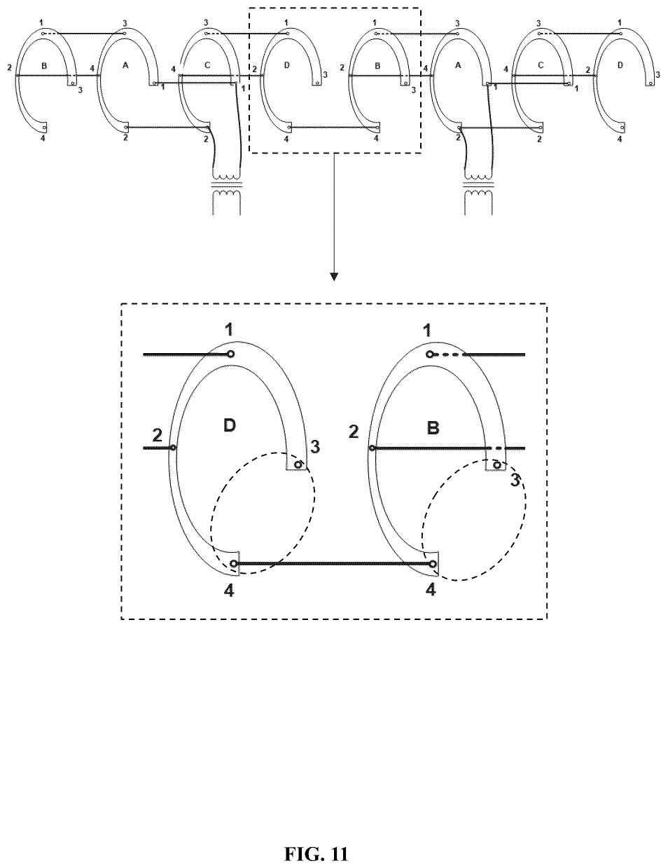

In some embodiments, the PCB may be disposed around the X-ray tube. In some embodiments, the PCB may be arc-shaped, ring-shaped, rectangle-shape, etc. Preferably, the PCB may be arc-shaped, e.g., ¾ ring-shaped, ⅔ ring-shaped, ⅘ ring-shaped, etc. More descriptions regarding the arc-shaped PCB may be found in in FIG. 10 or FIG. 11.

In some embodiments, the rectifier filter circuit may include at least one of a full bridge rectifier filter circuit or a bipolar CW circuit. For example, the bipolar CW circuit may include a two-stage bipolar CW circuit, a three-stage bipolar CW circuit, or a four-stage bipolar CW circuit.

In some embodiments, as illustrated in FIG. 3, the high voltage generator 310 may further include the filament transformer 315, e.g., the filament transformer may be also located in the housing of the X-ray device. The filament transformer may be configured to isolate a DC high voltage and amplify a current loaded into a filament of the X-ray bulb. A primary side of the filament transformer may be connected to a filament control unit disposed outside of the housing, and a secondary side of the filament transformer may be connected to a filament in the X-ray tube. A voltage output from the secondary side of the filament transformer may be used to power the filament in the X-ray tube, so that the filament may heat up and generate electrons at the cathode, for example, as shown in FIG. 5 or FIG. 6.

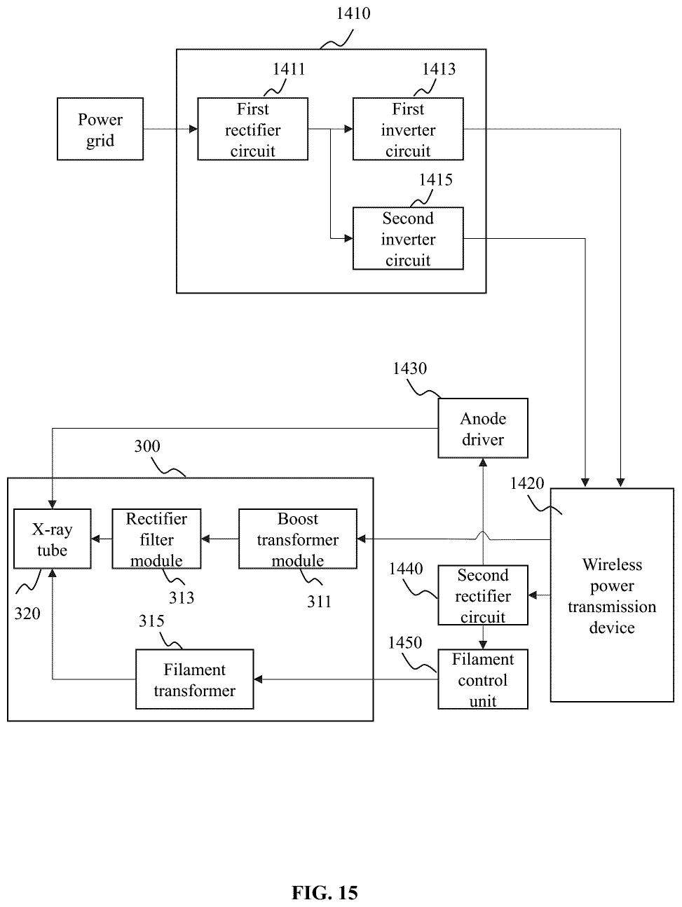

The filament control unit may be connected through a wiring panel of the X-ray device, thereby connecting to the filament transformer inside the X-ray device, as shown in FIG. 15. In this embodiment, the filament control unit may be located outside of the X-ray device. In some embodiments, the filament control unit may also be located inside the housing of the X-ray device. In some embodiments, the filament control unit may adopt a dual half bridge circuit or a full bridge circuit.

In some implementations, the filament transformer may include a dual filament transformer or a single filament transformer. For example, the dual filament transformer may be used when the X-ray tube is bipolar, and the single filament transformer may be used when the X-ray tube is unipolar.

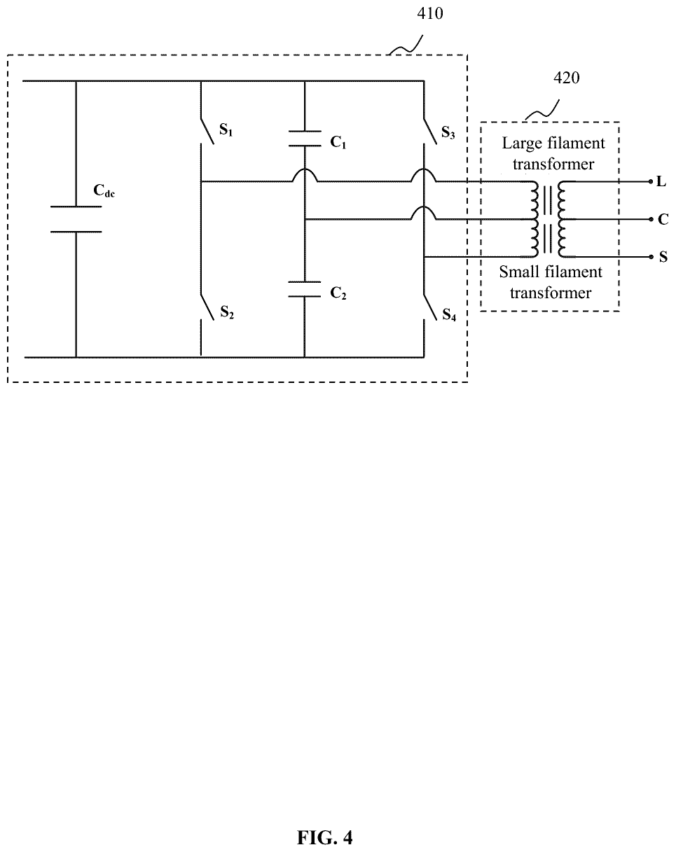

Taking the bipolar tube as an example, as shown in FIG. 4, a filament transformer 420 may include a large filament transformer and a small filament transformer. The filament control unit 410 may adopt the dual half bridge circuit. Output ends of the dual half bridge circuit may be connected to the large filament transformer and small filament transformer, respectively. A voltage output from the large filament transformer may power a large filament (e.g., an anode filament of the X-ray tube), and a voltage output from the small filament transformer may power a small filament (e.g., an cathode filament of the X-ray tube).

In some embodiments, the filament transformer and/or boost transformer module may include a planar transformer structure. In some embodiments, a magnetic core of the planar transformer may include a magnetic core of a flat structure. In some embodiments, the magnetic core may include a power magnetic core or a high conductivity magnetic core. For example, the magnetic core may include an EI, PEE, PEI, ER, ETD, EQ/EQI, EP, EFD, or EPC power magnetic core, or a U, ET, FT high conductivity magnetic core.

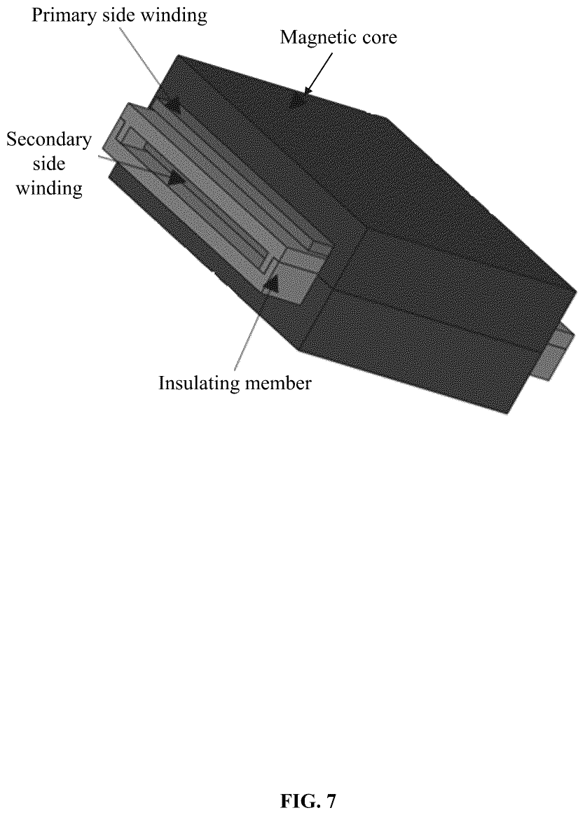

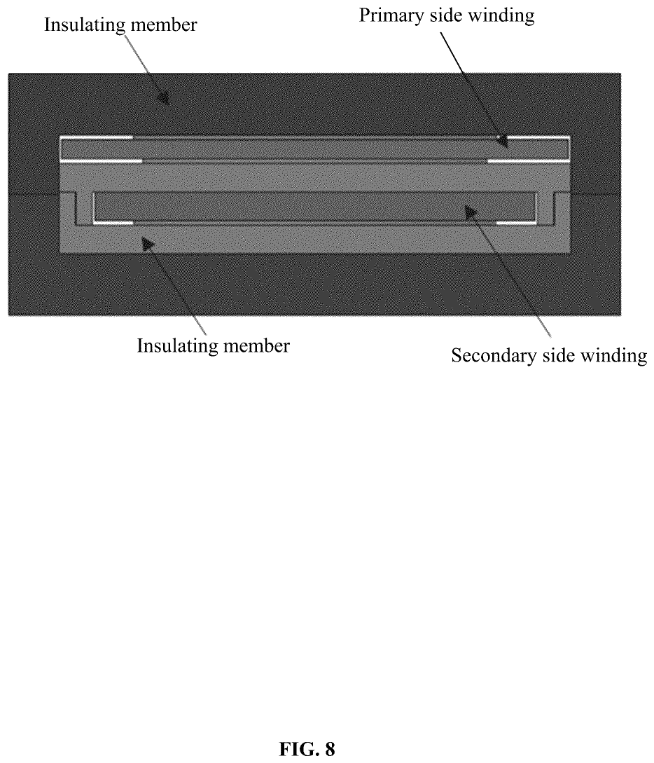

As described in connection with FIG. 7 and FIG. 8, FIG. 7 is a schematic diagram illustrating an exemplary three-dimensional structure of a planar transformer according some embodiments of the present disclosure, and FIG. 8 is a side view of a planar transformer according some embodiments of the present disclosure. Taking an ER magnetic core as an example, as shown in FIG. 7 or FIG. 8, a primary side winding and a secondary side winding may be disposed in the form of wire-wrapped wires or a PCB winding inside a window of the ER magnetic core. A solid insulating member may be used between the primary side winding and the secondary side winding and between the secondary side winding and the magnetic core for insulation.

The filament transformer or the boost transformer may include the planar transformer structure, which may reduce a volume of the transformer, so that the boost transformer module, the rectifier filter module, and the X-ray tube may be more tightly integrated into the housing of the X-ray device, and a volume of the X-ray device may be reduced while a contour state of the X-ray tube is kept.

The X-ray tube may generate X-rays under the control of the high voltage generator. The X-rays may be emitted towards a target object to obtain scanning data of the target object. For example, the high voltage generator 310 may power the cathode filament of the X-ray tube 320, so that the filament may heat up and generate electrons at the cathode; the electrons may impact a target disk at the anode at a high speed under the control of a high-voltage electric field to generate the X-rays, and emit the X-rays towards the target object; a detector may receive the X-rays that pass through the target object, thereby obtaining the scanning data of the target object.

In some embodiments, a shielding structure used to shield X-rays may be disposed on an outer side of a tube core in the X-ray tube. For example, as shown in FIG. 5, the tube core 527 of the X-ray tube may be wrapped with a lead skin used to shield from X-rays.

The shielding structure may be disposed on the outer side of the tube core in the X-ray tube, which may not only prevent X-rays from leaking out from a periphery of the housing of the X-ray device, but also avoid a problem such as component aging and failure caused by long-term exposure of a high-voltage circuit to X-ray radiation, thereby improving the service life of the device (e.g., the X-ray device, the imaging equipment including the X-ray device).

In some embodiments, a first insulating structure may be disposed between the X-ray tube and the rectifier filter module. In some embodiments, the first insulating structure may include a solid insulating member, an insulating oil, or an insulating gas. In some embodiments, each rectifier filter circuit of the rectifier filter module may be disposed between two insulating members. For example, each rectifier filter circuit of the rectifier filter module may be disposed between the two insulating structures, forming a structure (e.g., the insulating structure—the rectifier filter circuit—the insulating structure). The structure may surround the cathode or the anode of the X-ray tube 320. In some embodiments, each PCB corresponding to the rectifier filter module may be disposed between the two insulating members. For example, as described above, the first PCB is disposed between the first insulating member and the second insulating member, and the second PCB is disposed between the third insulating member and the fourth insulating member.

The first insulating structure may be disposed between the rectifier filter module and the X-ray tube, which may improve the insulation performance between the X-ray tube and the rectifier filter module.

In some embodiments, the X-ray device 300 may further include a sampling circuit, which may be used to sample a current and/or a voltage at the anode or the cathode of the X-ray tube.

In some embodiments, when the X-ray tube is bipolar, as shown in FIG. 5, the X-ray device may include two sets of sampling circuits 541 and 543. The two sets of sampling circuits 541 and 543 may be used to sample the current and the voltage of the anode and the cathode of the X-ray tube, respectively. In the sampling circuit 541 connected to the anode, a sampling resistor may be disposed between a ground terminal D and the rectifier filter circuit, which may be used to realize sampling of the current of the anode (e.g., Y2); and the sampling resistor and a sampling capacitor connected in parallel may be disposed between the ground terminal D and the anode, which may be used to realize sampling of the voltage of the anode (e.g., Y1). Similarly, in the sampling circuit 543 connected to the cathode, a sampling resistor may be disposed between the ground terminal D and the rectifier filter circuit, which may be used to realize sampling of the current of the cathode (e.g., Y3); and the sampling resistor and a sampling capacitor connected in parallel may be disposed between the ground terminal D and the cathode, which may be used to realize sampling of the voltage of the cathode (e.g., Y4).

In some embodiments, when the X-ray tube is unipolar, as shown in FIG. 6, the X-ray device may include a sampling circuit 640 used to sample the current and/or the voltage at the anode of the X-ray tube. A sampling principle of the unipolar X-ray tube may be similar to that of the bipolar X-ray tube, and more descriptions may be found in the relevant descriptions of FIG. 5, which is not repeated here.

In some embodiments, the sampling circuit may include a high voltage sampling resistor and a low voltage sampling resistor. A resistor in parallel with the capacitor may be used for low voltage sampling and a resistor in series with the capacitor may be used for high voltage sampling. For example, as shown in FIG. 9(a), a resistor corresponding to an Y1 end and in parallel with the capacitor may be used for low voltage sampling at an anode of an X-ray tube 920, and a resistor 955 corresponding to the Y1 end and in series with the capacitor may be used for high voltage sampling at an anode of the X-ray tube 920. Similarly, a resistor corresponding to an Y4 end and in parallel with the capacitor may be used for low voltage sampling at the cathode of the X-ray tube 920, and a resistor 957 corresponding to the Y4 end and in series with the capacitor may be used for high voltage sampling at the cathode of the X-ray tube 920.

In some embodiments, an X-ray device 300 may include a suppression resistor Rarc. In some embodiments, the resistor Rarc may be connected between the anode of the X-ray tube and the rectifier filter module. For example, as shown in FIG. 9(a), a resistor 951 may be connected between the anode of the X-ray tube 920 and a rectifier filter module 913. The resistor 951 may be used to perform ignition suppression on the anode of the X-ray tube to avoid damage to a front-end device of the X-ray tube caused by a short circuit.

In some embodiments, a high voltage sampling resistor Ras and/or the resistor Rarc may adopt a thin sheet high voltage resistor. For example, resistors 951, 953, 955, and 957 in FIG. 9(a) may all adopt thin sheet high voltage resistors. In some embodiments, the high voltage sampling resistor Ras and the suppression resistor Rarc may be disposed parallel to an edge of each PCB along a stacking direction of the PCBs, for example, as shown in FIG. 13. In some embodiments, the high voltage sampling resistor Ras and the suppression resistor Rarc may be fixed inside insulating members of the PCBs. For example, the resistor 951 and the resistor 955 may be fixed inside a first insulating member and a second insulating member corresponding to a first PCB, and the resistor 953 and the resistor 957 may be fixed inside a third insulating member and a fourth insulating member corresponding to a second PCB.

The thin sheet high voltage resistor may be adopted, which may make the resistor more tightly disposed inside the X-ray device and reduce the volume of the X-ray device. The high voltage sampling resistor Ras and the suppression resistor Rarc may be fixed inside the insulating members, which may improve the insulation performance and improve the service life.

In the X-ray device provided in the embodiment, the boost transformer module, the rectifier filter module, the filament transformer, and the X-ray tube may be disposed in the same housing, and the housing may be filled with the insulating material, so that the boost transformer module, the rectifier filter module, the filament transformer, and X-ray tube may be in the same insulating environment. Therefore, merely an ordinary connection device may need to be used between the high voltage oil tank including the boost transformer module and the rectifier filter module and the X-ray tube, and there may be no need to use the high-voltage connection device such as the high-voltage plug, the high-voltage socket, the high-voltage cable, etc., which may reduce the complexity of the high-voltage connection and save the cost of the high-voltage connection.

Additionally, if the above X-ray device is applied to the DR device, an appearance of the DR device may be more concise, which may be convenient for movement operation of the DR system. In addition, if the above X-ray device is applied to the CT device, a count and weights of the components of the rotating gantry of the CT device may be reduced, and the wear and tear of the bearing of the rotating gantry may be slowed down, which may be conducive to increasing the speed of the rotating gantry, thereby improving a temporal resolution of the scanning of the CT device.

It should be noted that the above descriptions of the X-ray device 300 is provided merely for the purpose of illustration and is not intended to limit the scope of the present disclosure. For those skilled in the art, various modifications and changes may be made to the process under the guidance of the present disclosure. However, these changes and modifications do not depart from the scope of the present disclosure.

FIG. 5 is a schematic diagram illustrating an exemplary circuit structure of an X-ray device corresponding to a bipolar tube according to some embodiments of the present disclosure. FIG. 5 is illustrated by taking a boost transformer module including four boost transformers and a rectifier filter module including four rectifier filter circuits as an example.

In some embodiments, as shown in FIG. 5, the boost transformer module 511, the rectifier filter module 513, a filament transformer 515, and an X-ray tube 520 may be disposed in a housing 510 of the X-ray device. The housing 510 may be filled with an insulating material, and a filament control unit 560 may be disposed outside the housing 510. The boost transformer module 511 may include the four boost transformers, and primary sides (left side of the boost transformer module 511) of the four boost transformers may be connected in parallel. Input ends A and B of the boost transformer module 511 may be used to receive a high-frequency AC voltage and increase the received voltage. The rectifier filter module 513 may include the four rectifier filter circuits. A secondary side (right side of the boost transformer module 511) of each boost transformer may be respectively connected to the input ends of the rectifier filter circuit corresponding to the boost transformer. Output ends of the four rectifier filter circuits may be connected in series. The rectifier filter module 513 may output a DC voltage by rectifying and filtering the increased AC voltage of the boost transformer module 511 and apply the DC voltage to the X-ray tube 520 to generate a high-voltage electric field between an anode and an cathode of the X-ray tube 520. At the same time, under the control of the filament control unit 560, a voltage output from the secondary side of the filament transformer may power a filament of the cathode of the X-ray tube 520, so that the filament may heat up and generate an electron beam S at the cathode. The electron beam S may impact a target disk of the anode at a high speed under the control of the high-voltage electric field to generate X-rays 521, which may be emitted through a ray window 523. In some embodiments, the anode driver 550 may provide an anode drive power supply to power a motor of the anode of the X-ray tube 520, which may cause the target disk of the anode to rotate at high speed and facilitate heat dissipation of the target disk of the anode.

As described above, when the X-ray tube is bipolar, an intermediate contact of all the rectifier filter circuits may be connected to a ground terminal. One of series-connected output ends of all the rectifier filter circuits may be connected to the anode of the X-ray tube and another end of the series-connected output ends of all the rectifier filter circuits may be connected to the cathode of the X-ray tube. In FIG. 5, the intermediate contact of all the rectifier filter circuits may be a midpoint of two middle rectifier filter circuits connected in series. As shown in FIG. 5, the intermediate contact of the rectifier filter circuits may be connected to the ground terminal D. One end of an uppermost rectifier filter circuit may be connected to the anode of the X-ray tube 520, and another end of a lowermost rectifier filter circuit may be connected to the cathode of the X-ray tube 520. A voltage of the anode may be capable of reaching +70 kV, and a voltage of the cathode may be capable of reaching −70 kV.

FIG. 6 is a schematic diagram illustrating an exemplary circuit structure of an X-ray device corresponding to a unipolar tube according to some embodiments of the present disclosure. Similar to FIG. 5, FIG. 6 is illustrated by taking a boost transformer module including four boost transformers and a rectifier filter module including four rectifier filter circuits as example.

As shown in FIG. 6, the boost transformer module 611, the rectifier filter module 613, a filament transformer 615, and an X-ray tube 620 may be disposed in a housing 610 of the X-ray device. The boost transformer module 611 may include the four boost transformers, and primary sides (left side of the boost transformer module 611) of the four boost transformers may be connected in parallel. Input ends A and B of the boost transformer module 611 may be used to receive a high-frequency AC voltage and increase the received voltage. The rectifier filter module 613 may include the four rectifier filter circuits. A secondary side of each boost transformer (right side of the boost transformer module 611) may be respectively connected to the input ends of the rectifier filter circuit corresponding to the boost transformer. Output ends of the four rectifier filter circuits may be connected in series.

When the X-ray tube is unipolar, as shown in FIG. 6, one output end of an uppermost rectifier filter circuit of the rectifier filter module 613 may be connected to the anode of the X-ray tube 620, and another output end of a lowermost rectifier filter circuit of the rectifier filter module 613 may be connected to the cathode of the X-ray tube 620. In some embodiments, to facilitate heat dissipation of the anode of the X-ray tube, a cable connected to the anode may be grounded. At this time, there may be no insulating pressure near the anode, the X-ray tube may be unipolar, and a voltage of the cathode is capable of reaching −140 kV.

It should be noted that the above descriptions of FIGS. 5 and 6 is merely provided for the purpose of illustration and is not intended to limit the scope of the present disclosure. For those skilled in the art, various modifications and changes may be made to the process under the guidance of the present disclosure For example, the boost transformer module 611 in FIG. 6 may include one boost transformer, the one boost transformer may be connected to four rectifier filter circuits. As another example, the boost transformer module 511 in FIG. 5 may include two boost transformers, and each of the two boost transformers may be connected to two rectifier filter circuits, respectively. As yet another example, FIG. 5 or FIG. 6 may both include two boost transformers and two rectifier filter circuits. However, these variations and changes do not depart from the scope of the present disclosure.

FIG. 9(a) is a schematic diagrams illustrating an exemplary circuit structure of an X-ray device according to some embodiments of the present disclosure. FIG. 9(b) is a schematic diagrams illustrating an exemplary circuit structure of an X-ray device according to some embodiments of the present disclosure. Similarly to FIG. 5, FIGS. 9(a)-9(b) are illustrated by taking a bipolar X-ray tube as an example.

As shown in FIG. 9(a), in some embodiments, a boost transformer module 911 may include four boost transformers. A rectifier filter module 913 may include four rectifier filter circuits. The boost transformer module 911, the rectifier filter module 913, and an X-ray tube 920 may be disposed in a housing 910 filled with an insulating material. In some embodiments, each of the rectifier filter circuits may include a two-stage bipolar CW circuit. Primary sides (left side of the boost transformer module 911) of the four boost transformers may be connected in parallel, which may be used to receive a high-frequency AC voltage. A secondary side (right side of the boost transformer module 911) of each boost transformer may be respectively connected to input ends of the bipolar CW circuit corresponding to the boost transformer, which may be used to receive increased AC voltage.

As shown in FIG. 9(b), each of the bipolar CW circuits may include four basic units 961, 963, 965, and 967. In some embodiments, each basic unit may be mounted on a PCB to form a rectifier board. Each rectifier board may have four ports, e.g., ports 1 to 4 in FIG. 9(b).

As shown in FIG. 9(b), a connection of rectifier boards A˜D formed by the four basic units in the bipolar CW circuit may be taken as an example. Ports 1 and 2 of rectifier board B (e.g., the rectifier board formed by the basic unit 963) may be electrically connected to ports 3 and 4 of rectifier board A (e.g., the rectifier board formed by the basic unit 961), respectively. Ports 1 and 2 of rectifier board A may be electrically connected to ports 1 and 2 of rectifier board C (e.g., the rectifier board formed by the basic unit 965), respectively. Ports 3 and 4 of rectifier board C may be electrically connected to ports 1 and 2 of rectifier board D (e.g., the rectifier board formed by basic unit 967), respectively. Output ends of all bipolar CW circuits corresponding to an anode and a cathode of the X-ray tube 920 may be connected in series through ports 4.

In some embodiments, port 4 of rectifier board B in the bipolar CW circuit (e.g., an uppermost rectifier filter circuit in FIG. 9(a)) with a highest anode voltage may be connected to the anode of the X-ray tube 920 via a suppression resistor 951. In some embodiments, rectifier board D in the bipolar CW circuit (e.g., a second rectifier filter circuit from top to bottom in FIG. 9(a)) with a lowest anode voltage may be connected to the current sampling end Y2.

As shown in FIG. 9(a), one end of the resistor 955 may be connected to port 4 of rectifier board B in the bipolar CW circuit with the highest anode voltage; and another end of the resistor 955 may be connected to a voltage sampling resistor (Y1 end resistor) in a sampling circuit 941. Accordingly, one end of the resistor 957 may be connected to port 4 of a rectifier board in the bipolar CW circuit (e.g., a lowermost rectifier filter circuit in FIG. 9(a)) with a highest cathode voltage; and another end of the resistor 957 may be connected to a voltage sampling resistor (Y4 end resistor) in a sampling circuit 943. The resistor 955 and the resistor 957 may be used for high voltage sampling, and the resistors at the Y1 and Y4 ends may be used for low voltage sampling. In some embodiments, sampling boards of the sampling circuits 941 and 943 may be mounted outside of the housing 910 of the X-ray device.

It is understood that the above descriptions of FIGS. 9(a)-9(b) are merely provided for the purpose of illustration and is not intended to limit the scope of the present disclosure. For those skilled in the art, various modifications and changes may be made to the process under the guidance of the present disclosure For example, the boost transformer module 911 may include two boost transformers, and each of the two boost transformers may be connected to two rectifier filter circuits, respectively; or one of the two boost transformers may be connected to one rectifier filter circuit, and another of the of the two boost transformers may be connected to three rectifier filter circuits. As another example, the boost transformer module 911 may include two boost transformers, and the rectifier filter module 913 may include two sets of bipolar CW circuits. A secondary side of a first boost transformer may be connected to an input end of a first bipolar CW circuit, and a secondary side of a second boost transformer may be connected to an input end of a second bipolar CW circuit. A primary side of the first boost transformer may be connected to a primary side of the second boost transformer in parallel, and an output end of the first bipolar CW circuit may be connected to an output end of the second bipolar CW circuit in series. One of the series-connected output ends may be connected to the cathode of the X-ray tube 920 and another of the series-connected output ends may be connected to the anode of the X-ray tube 920. However, these changes and modifications do not depart from the scope of the present disclosure.

FIG. 10 is a schematic diagram illustrating an exemplary structure of a rectifier board according to some embodiments of the present disclosure.

As described above, in some embodiments, a PCB may be arc-shaped. The PCB provided with a rectifier filter circuit may be also referred to as a rectifier board. FIG. 10 shows a schematic structure of a rectifier board with a ¾ ring-shaped PCB by taking a bipolar CW circuit with a two-stage rectifier filter circuit as an example.

In some embodiments, as shown in FIGS. 10(a)˜(d), four basic units of the bipolar CW circuit may be respectively disposed on one arc shaped PCB, which may form four rectifier boards A, B, C, and D, and four ports 1, 2, 3, and 4 of each basic unit may be evenly and symmetrically distributed on the arc-shaped PCB corresponding to the basic unit. In some embodiments, as shown in FIGS. 10(a)˜(d), in each of the four rectifier boards, all diodes 1010 may be connected in series using a plurality of surface-mounted diodes; all capacitors 1020 may be connected in series using a plurality of surface-mounted multilayer ceramic capacitors, and the diodes 1010 and the capacitors 1020 may be distributed between four ports 1, 2, 3, and 4 of the rectifier board.

It is understood that the above descriptions of the arc-shaped rectifier board is merely provided for the purpose of illustration and is not intended to limit the scope of the present disclosure. For those skilled in the art, various modifications and changes may be made to the process under the guidance of the present disclosure. For example, basic units A and B of the bipolar CW circuit may be disposed on PCB A, and basic units C and D may be disposed on PCB B. Alternatively, basic units A˜D may be disposed on PCB A. As another example, the rectifier filter circuit may be a three-stage CW circuit. Accordingly, the rectifier filter circuit may include six basic units, and the six basic units may be disposed on different PCBs. The four ports of each unit may be evenly distributed on the arc-shaped PCB corresponding to the unit. However, these changes and modifications do not depart from the scope of the present disclosure.

FIG. 11 is a schematic diagram illustrating an exemplary connection structure between a rectifier board and a boost transformer according some embodiments of the present disclosure. As described above, in some embodiments, two or more PCBs may be stacked. FIG. 11 illustrates a schematic diagram of the connection structure between two rectifier filter circuits and the boost transformer by taking a bipolar CW circuit with a two-stage rectifier filter circuits as an example.

In some embodiments, as shown in FIG. 11, four rectifier boards (e.g., four rectifier boards B, A, C, and D on the left side of the arrow) corresponding to a first rectifier filter circuit, and four rectifier boards (e.g., four rectifier boards B, A, C, and D on the right side of the arrow) corresponding to a second rectifier filter circuit may be all stacked. Two rectifier boards connected to the first rectifier filter circuit and the second rectifier filter circuit may be connected in series, for example, as shown in the rectangular dashed box.

As described above, each stacked rectifier may be snapped between an inner insulating member and an outer insulating member. In some embodiments, a ¾ ring-shaped region of each rectifier board may surround a cathode and/or an anode of the X-ray tube together with the insulating members. For example, as shown in FIG. 12 or FIG. 13, the four rectifier boards of the first rectifier filter circuit may surround the cathode of the X-ray tube together with the insulating members, and the four rectifier boards of the second rectifier filter circuit may surround the anode of the X-ray tube together with the insulating members. As another example, the four rectifier boards of the first rectifier filter circuit and the four rectifier boards of the second rectifier filter circuit may surround the cathode of the X-ray tube together with the insulating members.

In some embodiments, a missing ¼ region (e.g., the oval dashed box region in FIG. 11) of the rectifier board may be mounted with the boost transformer. As shown in FIG. 11, the first rectifier filter circuit and the second rectifier filter circuit may be respectively connected to a boost transformer. A secondary side of each boost transformer may be connected to ports 1 and 2 of rectifier board A corresponding to the rectifier filter circuit corresponding to the boost transformer or to ports 1 and 2 of the rectifier board C corresponding to the rectifier filter circuit corresponding to the boost transformer.

It is understood that the above descriptions of the rectifier board is merely provided for the purpose of illustration and is not intended to limit the scope of the present disclosure. For those skilled in the art, various modifications and changes may be made to the process under the guidance of the present disclosure. However, these changes and modifications do not depart from the scope of the present disclosure.

FIGS. 12 and 13 are three-dimensional sectional schematic diagrams illustrating an internal structure of an X-ray device in different directions according to some embodiments of the present disclosure.

Taking a bipolar X-ray tube as an example, as shown in FIG. 12 or FIG. 13, a housing 1210 of the X-ray device may include the X-ray tube 1220, boost transformers 1211 and 1213, rectifier filter circuits 1215 and 1217, a filament transformer 1219, insulating members 1231 and 1235, an anode motor stator 1230, and sampling resistors 1241 and 1243. The rectifier filter circuits 1215 and 1217 may be disposed on arc-shaped PCBs. The boost transformer 1211 may be mounted in the middle of a missing region (e.g., the oval dashed box region in FIG. 11) of the PCB corresponding to the rectifier filter circuit 1215. The boost transformer 1213 may be mounted in the middle of the missing region of the PCB corresponding to the rectifier filter circuit 1217. The boost transformers 1211 and 1213 and the filament transformer 1219 may all adopt a planar structure transformer. The rectifier filter circuit 1215 may surround an anode of the X-ray tube 1220 together with the insulating member 1231, and the rectifier filter circuit 1217 may surround a cathode of the X-ray tube 1220 together with the insulating member 1235. The resistor 1241 may be used for high voltage sampling at the anode of the X-ray tube 1220, and the resistor 1243 may be used for high voltage sampling at the cathode of the X-ray tube 1220.

A high voltage generator may convert an AC low-voltage into a DC high-voltage through the boost transformers 1211 and 1213 and the rectifier filter circuits 1215 and 1217. At the same time, the high voltage generator may apply the DC high-voltage between the anode and cathode of the X-ray tube 1220, thereby generating a high-voltage electric field between the anode and cathode of the X-ray tube 1220. At the same time, the high voltage generator may power a cathode filament of the X-ray tube 1220 through the filament transformer 1219, so that the filament may heat up and generate free electrons at the cathode. The free electrons may impact an anode target disk of the X-ray tube 1220 at a high speed under the control of the high voltage electric field to generate X-rays, and the X-rays may be emitted from an X-ray window 1250.

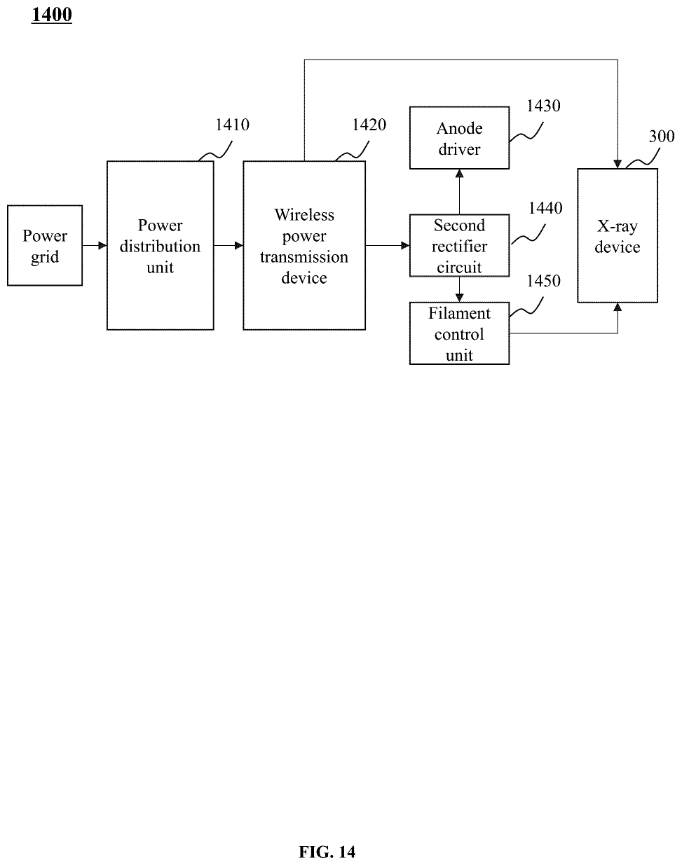

FIG. 14 is a block diagram illustrating an exemplary structure of an imaging device according to some embodiments of the present disclosure.

In embodiments of the present disclosure, the imaging device 1400 including the X-ray device 300 described above is also provided. In some embodiments, the imaging device may include the X-ray imaging device. For example, the imaging device may include a CT device, or a DR device.

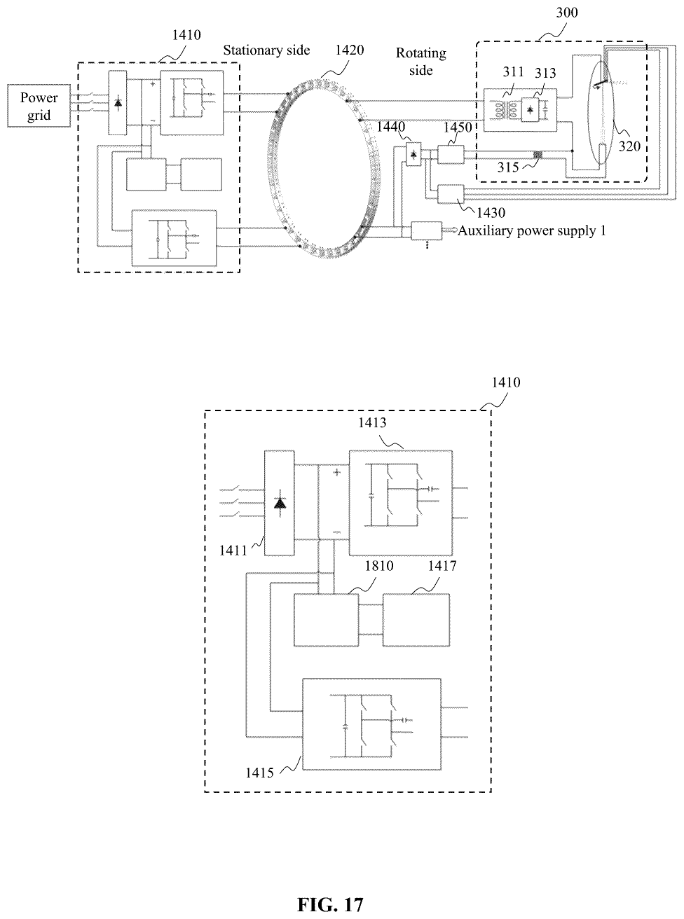

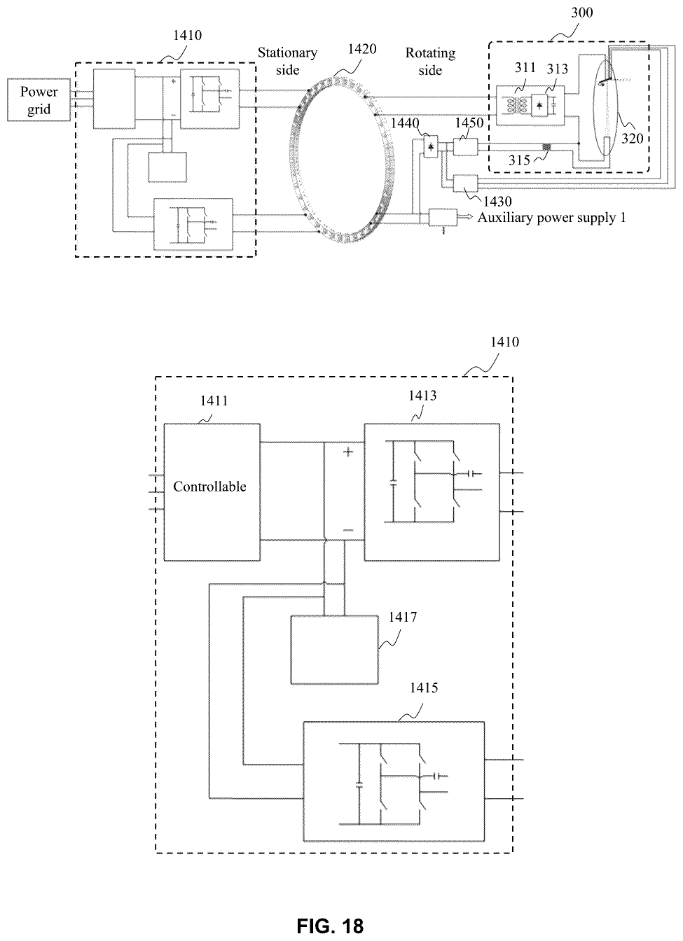

In some embodiments, as shown in FIG. 17 or FIG. 18, the imaging device 1400 may further include a gantry, and the X-ray device 300 may be disposed on a rotating side of the gantry.

In some embodiments, as shown in FIG. 14, the imaging device 1400 may also include a power distribution unit (PDU) 1410 and a wireless power transmission device 1420.

In some embodiments, the wireless power transmission device 1420 may include a main primary side winding and a main secondary side winding. The main primary side winding may be disposed on a stationary side of the gantry, and the main secondary side winding may be disposed on the rotating side of the gantry and correspond to the main primary side winding, for example, as shown in FIG. 17 or FIG. 18. In some embodiments, the wireless power transmission device 1420 may further include an auxiliary primary side winding and an auxiliary secondary side winding. The auxiliary primary side winding may be disposed on the stationary side of the gantry of the imaging device 1400, and the auxiliary secondary side winding may be disposed on the rotating side of the gantry and correspond to the auxiliary primary side winding, for example, as shown in FIG. 17 or FIG. 18. In some embodiments, the wireless power transmission device 1420 may adopt a resolver. The rotary transformer may be an electromagnetic sensor, a stator winding may act as a primary side of the rotary transformer and receive an excitation voltage, and a rotor winding may act as a secondary side of the rotary transformer and obtain an induced voltage through electromagnetic coupling, thereby realizing wireless transmission of power.

The power distribution unit 1410 may be located on the stationary side of the gantry of the imaging device, for example, as shown in FIG. 17 or FIG. 18. In some embodiments, as shown in FIG. 15, the power distribution unit 1410 may include a first rectifier circuit 1411 and a first inverter circuit 1413. An output end of the first rectifier circuit 1411 may be connected to an input end of the first inverter circuit 1413, and an output end of the first inverter circuit 1413 may be connected to the main primary side winding of the wireless power transmission device 1420. The main secondary side winding of the wireless power transmission device 1420 may be connected to an input end of the boost transformer module 311 in the X-ray device 300.

The first rectifier circuit 1411 may be configured to receive a first AC voltage provided by a power grid and rectify the first AC voltage to output a first DC voltage. The first inverter circuit 1413 may be used to convert the first DC voltage output from the first rectifier circuit 1411 into a second AC voltage and transmit the second AC voltage to the boost transformer module 311 through the main primary side winding and the main secondary side winding in the wireless power transmission device 1420.

In some embodiments, the first rectifier circuit may be an uncontrollable rectifier circuit, as shown in FIG. 17. In some embodiments, the first rectifier circuit may be a controllable rectifier circuit, as shown in FIG. 18.

In some embodiments, as shown in FIG. 14, the imaging device 1400 may also include a second rectifier circuit 1440 and a filament control unit 1450. In some embodiments, the second rectifier circuit 1440 and the filament control unit 1450 may be located on the rotating side of the gantry of the imaging device 1400, for example, as shown in FIG. 17 or FIG. 18. As shown in FIG. 15, an output end of the second rectifier circuit 1440 may be connected to an input of the filament control unit 1450. The output end of the filament control unit 1450 may be connected to a primary side winding of the filament transformer 315 in the X-ray device 300, and a secondary side winding of the filament transformer 315 may be connected to a filament in the X-ray tube 320.

The second rectifier circuit 1440 may be configured to receive a third AC voltage transmitted by the wireless power transmission device 1420 and rectify the third AC voltage to output a second DC voltage. The filament control unit 1450 may be configured to convert the second DC voltage output by the second rectifier circuit 1440 into a fourth AC voltage and load the fourth AC voltage to a primary side of the filament transformer 315 in the X-ray device 300, for example, as shown in FIG. 15.

In some embodiments, as shown in FIG. 15, the power distribution unit 1410 may further include a second inverter circuit 1415. The output end of the first rectifier circuit 1411 may be connected to an input end of the second inverter circuit 1415. The output end of the second inverter circuit 1415 may be connected to the auxiliary primary side winding of the wireless power transmission device 1420, and the auxiliary secondary side winding may be connected to an input end of the second rectifier circuit 1440.

The second inverter circuit 1415 may be configured to convert the first DC voltage output by the first rectifier circuit 1411 into the third AC voltage and transmit the third AC voltage to the second rectifier circuit 1440 through the auxiliary primary side winding and the auxiliary secondary side winding in the wireless power transmission device 1420.

In some embodiments, as shown in FIG. 17 or FIG. 18, the power distribution unit 1410 may further include an energy storage unit 1417, and the energy storage unit 1417 may be connected to the output end of the first rectifier circuit 1411. In some embodiments, the energy storage unit 1417 may include an energy storage battery. In some embodiments, a capacity and count of the energy storage batteries may be determined according to actual application requirements.

The demand for grid capacity for X-ray imaging device may be reduced by introducing the energy storage unit on a DC bus.

In some embodiments, as shown in FIG. 17, the power distribution unit 1410 may further include a bidirectional DC converter 1810, and the bidirectional DC converter 1810 may be connected in series between the first rectifier circuit 1411 and the energy storage unit 1417. In this case, the first rectifier circuit 1411 may be an uncontrollable rectifier circuit.

The bidirectional DC converter may also be referred to as a bidirectional DC-DC converter, which may be a device that an realize current conversion. The device may enable bidirectional flow of DC energy. During use, the current output by the bidirectional DC converter may be either positive or negative, and the energy generated may flow in both directions. That is, the current output by the bidirectional DC converter may flow either from an input side to an output side or from the output side to the input side.

As shown in the lower part of FIG. 17, the first rectifier circuit 1411 may be connected to the energy storage unit 1417 through the bidirectional DC converter 1810, and the energy storage unit 1417 may be connected to the first inverter circuit 1413 and the second inverter circuit 1415 through the bidirectional DC converter 1810, respectively. In this way, the energy storage unit 1417 may be charged using a DC bus voltage output by the first rectifier circuit 1411 by controlling the bidirectional DC converter 1810. Alternatively, the energy storage unit 1417 may power the first inverter circuit 1413 and the second inverter circuit 1415 by controlling the bidirectional DC converter 1810.

In some embodiments, a constant DC bus voltage may be maintained by controlling the bidirectional DC converter (e.g., the bidirectional DC converter 1810) when scanning a patient using the imaging device. At this point, the energy storage unit 1417 may power the entire imaging device.

In some embodiments, when the imaging device is in a standby state, the bidirectional DC converter may be controlled to charge the energy storage unit to ensure that the energy storage unit is able to continuously maintain high power scanning of the imaging device. At this point, the grid may only need to provide an average power of the imaging device, so the demand for grid capacity of the imaging device may be reduced.

In some embodiments, the first rectifier circuit 1411 may be a controllable rectifier circuit, as shown in FIG. 18. The energy storage unit 1417 may be charged by controlling the first rectifier circuit 1411, or the energy storage unit 1417 may power the first inverter circuit 1413 and the second inverter circuit 1415. In some embodiments, the controllable first rectifier circuit 1411 may be controlled to charge the energy storage unit 1417 when the imaging device is in the standby state. In some embodiments, when the patient is scanned using the imaging device, the controllable first rectifier circuit 1411 and the energy storage unit 1417 may be controlled to power the first inverter circuit 1413 and the second inverter circuit 1415. In this case, the grid may only need to provide the average power of the imaging device, so the demand for gird capacity of the imaging device may be reduced.

In some embodiments, as shown in FIG. 14, the imaging device 1400 may also include an anode driver 1430. The anode driver 1430 may be located on the rotating side of the gantry and connected in series between the second rectifier circuit 1440 and X-ray tube 320, for example, as shown in FIG. 17 or FIG. 18. The anode driver 1430 may be configured to convert the second DC voltage output by the second rectifier circuit 1440 into a fifth AC voltage and load the fifth AC voltage to a motor driving the anode to rotate in the X-ray tube 320.

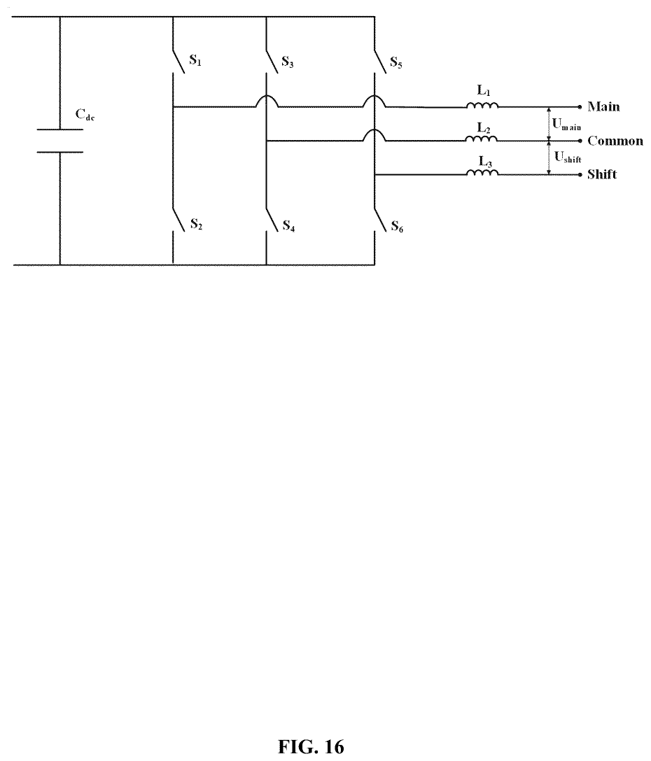

In some embodiments, the anode driver 1430 may adopt a three-phase H-bridge inverter circuit. As shown in FIG. 16, three outputs of the three-phase H-bridge inverter circuit may be connected to Main, Shift, and Common ends of a motor stator winding in the X-ray tube after filter inductors L1, L2, and L3, respectively. Two voltages Umain and Ushift output by the three-phase H-bridge inverter circuit may drive an anode target disk of the X-ray tube to rotate to help heat dissipation of the anode target disk of the X-ray tube. Frequencies of the voltages Umain and Ushift may be generally in a range of 50 to 180 Hz.

In some embodiments, the wireless power transmission device 1420 may include a main transmission channel and an auxiliary transmission channel. The main transmission channel may include the main primary side winding and the main secondary side winding, and the auxiliary transmission channel may include the auxiliary primary side winding and auxiliary secondary side winding. The first rectifier circuit 1411 may rectify the first AC voltage provided by the power grid into the first DC voltage. On the one hand, the first DC voltage may pass through the first inverter circuit 1413 and the first high-frequency AC voltage may be output. The first high-frequency AC voltage may be transmitted to the rotating side of the gantry through the main transmission channel of the wireless power transmission device 1420 to power the X-ray device 300. On the other hand, the first DC voltage may pass through the second inverter circuit 1415 and the second high-frequency AC voltage may be output. The second high-frequency AC voltage may be transmitted to the rotating side of the gantry through the auxiliary transmission channel of the wireless power transmission device 1420 to power the filament control unit 1450 and the anode driver 1430.

In some embodiments, the first DC voltage may be converted into an AC voltage corresponding to a scanning protocol by controlling the first inverter circuit 1413 differently based on the scanning protocol. In some embodiments, the voltage corresponding to the scanning protocol may be generated between the anode and the cathode of the X-ray tube through the transmission of the main transmission channel of the wireless power transmission device 1420 and the voltage conversion of the boost transformer and the rectifier filter circuit, thereby scanning a corresponding part of the patient.

It is understood that the above descriptions of the imaging device 1400 is merely provided for the purpose of illustration and is not intended to limit the scope of the present disclosure. For those skilled in the art, various modifications and changes to the process may be made under the guidance of the present disclosure. However, these changes and modifications do nor depart from the scope of the present disclosure.

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements and amendments to the present disclosure. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various parts of this specification are not necessarily all referring to the same embodiment. In addition, some features, structures, or features in the present disclosure of one or more embodiments may be appropriately combined.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that the present disclosure object requires more features than the features mentioned in the claims. Rather, claimed subject matter may lie in less than all features of a single foregoing disclosed embodiment.

In some embodiments, the numbers expressing quantities or properties used to describe and claim certain embodiments of the present disclosure are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. An X-ray device, comprising a high voltage generator and an X-ray tube, wherein the high voltage generator and the X-ray tube are disposed in a same housing, and the housing is filled with an insulating material;
the high voltage generator includes at least one of a boost transformer module and a rectifier filter module; and the boost transformer module and the rectifier filter module are disposed around the X-ray tube.

2. The X-ray device of claim 1, wherein
the boost transformer module is configured to receive an alternating current (AC) voltage and increase the AC voltage; and
the rectifier filter module is configured to output a direct current (DC) voltage by rectifying and filtering the increased AC voltage and apply the DC voltage to the X-ray tube.

3. The X-ray device of claim 2, wherein the boost transformer module includes at least one boost transformer, and the rectifier filter module includes at least one rectifier filter circuit.

4. The X-ray device of claim 3, wherein the boost transformer module includes at least two boost transformers, and the rectifier filter module includes at least two rectifier filter circuits.

5. The X-ray device of claim 4, wherein
primary sides of the at least two boost transformers are connected in parallel, and/or
secondary sides of the at least two boost transformers are connected in series.

6. The X-ray device of claim 5, wherein output ends of the at least two rectifier filter circuits are connected in series, and an intermediate contact of the at least two rectifier filtering circuits is connected to a ground terminal.

7. The X-ray device of claim 6, wherein
a first printed circuit board (PCB) surrounds an anode of the X-ray tube and a second PCB surrounds a cathode of the X-ray tube; and
the first PCB includes a PCB where a first rectifier filter circuit of the at least two rectifier filter circuits is located, the first rectifier filter circuit being connected in series between the anode and the ground terminal of the X-ray tube; and
the second PCB includes a PCB where a second rectifier filter circuit of the at least two rectifier filter circuits is located, the second rectifier filter circuit being connected in series between the cathode and the ground terminal of the X-ray tube.

8. The X-ray device of claim 3, wherein part or all of the at least one rectifier filter circuit is located on a PCB
disposed around the X-ray tube.

9. The X-ray device of claim 8, wherein the PCB is arc-shaped, and the boost transformer is mounted in a missing region of the arc-shaped PCB.

10. The X-ray device of claim 3, wherein the rectifier filter circuit includes at least one of a bipolar continuous wave (CW) circuit or a full bridge rectifier filter circuit.

11. The X-ray device of claim 10, wherein the high voltage generator further include a filament transformer, and the filament transformer is configured to isolate a DC high-voltage and amplify a current loaded into a filament of the X-ray bulb, and a primary side of the filament transformer is connected to a filament control unit disposed outside the housing, and a secondary side of the filament transformer is connected to a filament in the X-ray tube.

12. The X-ray device of claim 11, wherein the filament transformer and/or the boost transformer module includes a planar transformer structure.

13. The X-ray device of claim 1, further comprising at least two PCBs, and the at least two PCBs are stacked, and a second insulating structure is disposed between the at least two stacked PCBs.

14. The X-ray device of claim 1, wherein a shielding structure used to shield X-rays is disposed on an outer side of a tube core in the X-ray tube.

15. The X-ray device of claim 1, wherein no high-voltage cable is connected between the high voltage generator and the X-ray tube.

16. An imaging device, comprising an X-ray device, wherein the X-ray device includes a high voltage generator and an X-ray tube, the high voltage generator and the X-ray tube are disposed in a same housing, and the housing is filled with an insulating material, the high voltage generator includes at least one of a boost transformer module and a rectifier filter module; and the boost transformer module and the rectifier filter module are disposed around the X-ray tube.

17. The imaging device of claim 16, further comprising a gantry, a power distribution unit and a wireless power transmission device, wherein
the X-ray device is disposed on a rotating side of the gantry,
the power distribution unit is disposed on a stationary side of the gantry and includes a first rectifier circuit and a first inverter circuit;
the wireless power transmission device includes a main primary side winding and a main secondary side winding, the main primary side winding being disposed on the stationary side of the gantry, the main secondary side winding corresponding to the main primary side winding and being located on the rotating side of the gantry;
an output end of the first rectifier circuit is connected to an input end of the first inverter circuit, an output end of the first inverter circuit is connected to the main primary side winding, and the main secondary side winding is connected to an input end of a boost transformer module in the X-ray device;
the first rectifier circuit is configured to receive a first alternating current (AC) voltage provided by a power grid and rectify the first AC voltage to output a first direct current (DC) voltage; and
the first inverter circuit is configured to convert the first DC voltage output by the first rectifier circuit into a second AC voltage and transmit the second AC voltage to the boost transformer module through the main primary side winding and the main secondary side winding in the wireless power transmission device.

18. The imaging device of claim 17, wherein the power distribution unit further includes an energy storage unit, and the energy storage unit is connected to the output end of the first rectifier circuit.

19. The imaging device of claim 18, wherein
the first rectifier circuit is an uncontrollable rectifier circuit; and
the power distribution unit further includes a bidirectional DC converter, and the bidirectional DC converter is connected in series between the first rectifier circuit and the energy storage unit.

20. The imaging device of claim 18, further comprising a second rectifier circuit and a filament control unit, wherein
the second rectifier circuit and the filament control unit are located on the rotating side of the gantry;
the power distribution unit further includes a second inverter circuit;
the wireless power transmission device further includes an auxiliary primary side winding and an auxiliary secondary side winding, the auxiliary primary side winding being located on the stationary side of the gantry, the auxiliary secondary side winding corresponding to the auxiliary primary side winding and being located on the rotating side of the gantry;
the second inverter circuit is configured to convert the first DC voltage output by the first rectifier circuit into a third AC voltage and transmit the third AC voltage to the second rectifier circuit through the auxiliary primary side winding and the auxiliary secondary side winding in the wireless power transmission device;
the second rectifier circuit is configured to receive the third AC voltage transmitted by the wireless power transmission device and rectify the third AC voltage to output a second DC voltage; and
the filament control unit is configured to convert the second DC voltage output by the second rectifier circuit into a fourth AC voltage and load the fourth AC voltage to a primary side of a filament transformer.
